SEQUENCE LISTING (14 sequences)

SEQ ID NO: 1 - DNA, 726 bp, Artificial sequence
  anti-CD7 scFv
gaagtccaacttgttgaatcaggaggtggcttggtaaaacctgggggctccctcaaacttagctgcgcggcatccggcctcacttttagctcttacgcaatgtcatgggtgagacagacaccggaaaagcgccttgagtgggttgcaagcatatcaagtggtgggttcacatactatccagacagtgtaaaagggcggttcacgatttcaagagacaacgcaagaaacatactgtacctgcagatgagctctctccgatccgaagatacagctatgtattactgcgcgagagatgaggtacgcggttaccttgatgtctggggagcggggactacagtgacagtctccagtggctccacttccggtagcggcaagcctggtagttccgagggcagtaccaagggggacattcagatgacgcagacgacttcctcactttcagccagtcttggtgatcgcgtcactataagttgttctgcatcacaaggtatatctaattacctgaattggtatcaacaaaagcccgacggcactgtcaagttgttgatatattacacctcctcactccattctggggtcccgtcccgattcagtggtagcgggtctggaacggactactccctcaccatttcaaacttggagcccgaggatattgctacctactattgtcagcagtactcaaaactgccttacacctttggagggggcacaaaactcgagataaaa

SEQ ID NO: 2 - protein, 242 aa, Artificial sequence
  anti-CD7 scFv
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSGSTSGSGKPGSSEGSTKGDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIK

SEQ ID NO: 3 - DNA, 1680 bp, Artificial sequence
  CAR
atgctcctgcttgtaacgagcctcctgctctgtgagctgcctcaccccgctttccttcttataccggaagtccaacttgttgaatcaggaggtggcttggtaaaacctgggggctccctcaaacttagctgcgcggcatccggcctcacttttagctcttacgcaatgtcatgggtgagacagacaccggaaaagcgccttgagtgggttgcaagcatatcaagtggtgggttcacatactatccagacagtgtaaaagggcggttcacgatttcaagagacaacgcaagaaacatactgtacctgcagatgagctctctccgatccgaagatacagctatgtattactgcgcgagagatgaggtacgcggttaccttgatgtctggggagcggggactacagtgacagtctccagtggctccacttccggtagcggcaagcctggtagttccgagggcagtaccaagggggacattcagatgacgcagacgacttcctcactttcagccagtcttggtgatcgcgtcactataagttgttctgcatcacaaggtatatctaattacctgaattggtatcaacaaaagcccgacggcactgtcaagttgttgatatattacacctcctcactccattctggggtcccgtcccgattcagtggtagcgggtctggaacggactactccctcaccatttcaaacttggagcccgaggatattgctacctactattgtcagcagtactcaaaactgccttacacctttggagggggcacaaaactcgagataaaagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccctgggcccacccgcaagcattaccagccctatgccccaccacgcgacttcgcagcctatcgctccgctagcggcggaggtggctctggcggtggcggatcagttgttaaacggggcagaaagaaactcctgtatatattcaaacaaccatttatgagaccagtacaaactactcaagaggaagatggctgtagctgccgatttccagaagaagaagaaggaggatgtgaactgggcggcggcggcagcggaggcggtggcagcggcggcggcggctctagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagttaa

SEQ ID NO: 4 - DNA, 2946 bp, Artificial sequence
  CAR
atgctcctgcttgtaacgagcctcctgctctgtgagctgcctcaccccgctttccttcttataccggaagtccaacttgttgaatcaggaggtggcttggtaaaacctgggggctccctcaaacttagctgcgcggcatccggcctcacttttagctcttacgcaatgtcatgggtgagacagacaccggaaaagcgccttgagtgggttgcaagcatatcaagtggtgggttcacatactatccagacagtgtaaaagggcggttcacgatttcaagagacaacgcaagaaacatactgtacctgcagatgagctctctccgatccgaagatacagctatgtattactgcgcgagagatgaggtacgcggttaccttgatgtctggggagcggggactacagtgacagtctccagtggctccacttccggtagcggcaagcctggtagttccgagggcagtaccaagggggacattcagatgacgcagacgacttcctcactttcagccagtcttggtgatcgcgtcactataagttgttctgcatcacaaggtatatctaattacctgaattggtatcaacaaaagcccgacggcactgtcaagttgttgatatattacacctcctcactccattctggggtcccgtcccgattcagtggtagcgggtctggaacggactactccctcaccatttcaaacttggagcccgaggatattgctacctactattgtcagcagtactcaaaactgccttacacctttggagggggcacaaaactcgagataaaagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccctgggcccacccgcaagcattaccagccctatgccccaccacgcgacttcgcagcctatcgctccgctagcggaggtggaggttctggaggtggtggaagtcaaagaaggaagtaccgcagcaacaaaggagaatctcccgtcgagccagccgagccctgtcattattcatgcccaagggaggaggagggaagtacaatcccaattcaagaagactacaggaagcccgaacctgcatgcagtccaggtggaggcggttctggaggcggtggctcccgggtgaaattctcacggtctgcagacgcacccgcttaccagcaaggccagaaccaactctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagtggctccggagccacgaacttctctctgttaaagcaagcaggagacgtggaagaaaaccccggtcccatgggagtgcaggtggaaaccatctccccaggagacgggcgcaccttccccaagcgcggccagacctgcgtggtgcactacaccgggatgcttgaagatggaaagaaagtggactcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaggtggaggcggttcaggcggcggcggcagcggcgccatggtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagttaa

SEQ ID NO: 5 - DNA, 1644 bp, Artificial sequence
  CAR
atgctcctgcttgtaacgagcctcctgctctgtgagctgcctcaccccgctttccttcttataccggaagtccaacttgttgaatcaggaggtggcttggtaaaacctgggggctccctcaaacttagctgcgcggcatccggcctcacttttagctcttacgcaatgtcatgggtgagacagacaccggaaaagcgccttgagtgggttgcaagcatatcaagtggtgggttcacatactatccagacagtgtaaaagggcggttcacgatttcaagagacaacgcaagaaacatactgtacctgcagatgagctctctccgatccgaagatacagctatgtattactgcgcgagagatgaggtacgcggttaccttgatgtctggggagcggggactacagtgacagtctccagtggctccacttccggtagcggcaagcctggtagttccgagggcagtaccaagggggacattcagatgacgcagacgacttcctcactttcagccagtcttggtgatcgcgtcactataagttgttctgcatcacaaggtatatctaattacctgaattggtatcaacaaaagcccgacggcactgtcaagttgttgatatattacacctcctcactccattctggggtcccgtcccgattcagtggtagcgggtctggaacggactactccctcaccatttcaaacttggagcccgaggatattgctacctactattgtcagcagtactcaaaactgccttacacctttggagggggcacaaaactcgagataaaagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccctgggcccacccgcaagcattaccagccctatgccccaccacgcgacttcgcagcctatcgctccgctagcggtggaggtggatctggtggtggaggttccaaatctcggcagacaccacctctcgcatctgtcgagatggaggccatggaggcactgcctgtcacatggggtacttccagcagggacgaggacctcgaaaattgctcccatcatctgggtggcggcggttccggtggtggaggttctcgggtgaaattctccaggtccgcagacgctccagcctaccagcaaggacagaaccaactgtacaatgagctgaatctgggaagacgcgaggagtatgacgtgctggacaaacggagaggtcgcgaccccgagatgggaggcaagcctcgcaggaagaacccacaggaaggactgtacaacgaactgcagaaggacaagatggccgaggcatactcagagatcggaatgaagggcgagagacgcagagggaaaggccacgacggactgtaccagggactgtcaactgccacaaaggatacttacgatgccctgcacatgcaggcactccctccacgctga

SEQ ID NO: 6 - protein, 559 aa, Artificial sequence
  CAR
MLLLVTSLLLCELPHPAFLLIPEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSGSTSGSGKPGSSEGSTKGDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSASGGGGSGGGGSVVKRGRKKLLYIFKQPFMRPVQTTQEEDGCSCRFPEEEEGGCELGGGGSGGGGSGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTS

SEQ ID NO: 7 - protein, 981 aa, Artificial sequence
  CAR
MLLLVTSLLLCELPHPAFLLIPEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSGSTSGSGKPGSSEGSTKGDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSASGGGGSGGGGSQRRKYRSNKGESPVEPAEPCHYSCPREEEGSTIPIQEDYRKPEPACSPGGGGSGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSGGGGSGAMVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 8 - protein, 547 aa, Artificial sequence
  CAR
MLLLVTSLLLCELPHPAFLLIPEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSGSTSGSGKPGSSEGSTKGDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSASGGGGSGGGGSKSRQTPPLASVEMEAMEALPVTWGTSSRDEDLENCSHHLGGGGSGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 1695 bp, Artificial sequence
  CAR
atgctcctgcttgtgacctcactcttgctttgtgaactgccacatccggcctttctgttgatcccgcaagtccaacttgttgagtcaggaggcggtctcgtgcagccaggtggatcacgcaaactttcttgtgcagcttccggattcacattcagtagtttcgggatgcattgggtcaggcaagctccagagaaagggctcgaatgggtagcgtatatctctagtggatctagtacgctgcattacgctgacaccgtcaaaggtcggtttacgattagcagggataaccccaagaatacgctgttcttgcagatgacatcattgcggtctgaagatacggccatgtactactgcgcccggtgggggaactatccccattatgcgatggattactggggtcaaggcacctctgtcacggtttcttccggaagcacgagtgggagtgggaaacctggcagctctgaggggagtacgaaaggtgatatagtgatgacacaaagccccgcctcacttgcagtgtctcttggggacagagcgaccatcagttgccgagccagcaaatctgtttctgcctctggatacagttatatgcactggtatcaacagaaaccggggcaaccgccaaagttgctcatatacttggcgtcaaatctcgaatccggtgttcctgcacggttctccggctctggatctggtacagatttcactctcaacatccatcccgtcgaggaggaggacgctgtaacctactactgtcagcattccagggagcttccttacacatttggcggcggcacaaagctcgagattaaggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccctgggcccacccgcaagcattaccagccctatgccccaccacgcgacttcgcagcctatcgctccgctagcggaggtggaggttctggaggtggtggaagtcaaagaaggaagtaccgcagcaacaaaggagaatctcccgtcgagccagccgagccctgtcattattcatgcccaagggaggaggagggaagtacaatcccaattcaagaagactacaggaagcccgaacctgcatgcagtccaggtggaggcggttctggaggcggtggctcccgggtgaaattctcacggtctgcagacgcacccgcttaccagcaaggccagaaccaactctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagt

SEQ ID NO: 10 - DNA, 1701 bp, Artificial sequence
  CAR
atgctcctgcttgtgacctcactcttgctttgtgaactgccacatccggcctttctgttgatcccgcaagtccaacttgttgagtcaggaggcggtctcgtgcagccaggtggatcacgcaaactttcttgtgcagcttccggattcacattcagtagtttcgggatgcattgggtcaggcaagctccagagaaagggctcgaatgggtagcgtatatctctagtggatctagtacgctgcattacgctgacaccgtcaaaggtcggtttacgattagcagggataaccccaagaatacgctgttcttgcagatgacatcattgcggtctgaagatacggccatgtactactgcgcccggtgggggaactatccccattatgcgatggattactggggtcaaggcacctctgtcacggtttcttccggaagcacgagtgggagtgggaaacctggcagctctgaggggagtacgaaaggtgatatagtgatgacacaaagccccgcctcacttgcagtgtctcttggggacagagcgaccatcagttgccgagccagcaaatctgtttctgcctctggatacagttatatgcactggtatcaacagaaaccggggcaaccgccaaagttgctcatatacttggcgtcaaatctcgaatccggtgttcctgcacggttctccggctctggatctggtacagatttcactctcaacatccatcccgtcgaggaggaggacgctgtaacctactactgtcagcattccagggagcttccttacacatttggcggcggcacaaagctcgagattaaggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccctgggcccacccgcaagcattaccagccctatgccccaccacgcgacttcgcagcctatcgctccgctagcggcggaggtggctctggcggtggcggatcagttgttaaacggggcagaaagaaactcctgtatatattcaaacaaccatttatgagaccagtacaaactactcaagaggaagatggctgtagctgccgatttccagaagaagaagaaggaggatgtgaactgggcggcggcggcagcggaggcggtggcagcggcggcggcggctctagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagttaa

SEQ ID NO: 11 - protein, 21 aa, Artificial sequence
  CD8a signal peptide
MALPVTALLLPLALLLHAARP

SEQ ID NO: 12 - protein, 22 aa, Artificial sequence
  GM-CSFR signal peptide
MLLLVTSLLLCELPHPAFLLIP

SEQ ID NO: 13 - protein, 5 aa, Artificial sequence
  hinge
GGGGS

SEQ ID NO: 14 - protein, 10 aa, Artificial sequence
  hinge
GGGGSGGGGS

(19) United States
(12) Patent Application Publication
(10) Pub. No.: US 2025/0222026 A1
(43) Pub. Date: Jul. 10, 2025

(54) CD7-BASED HUMANIZED CHIMERIC ANTIGEN RECEPTOR AND USE THEREOF

(71) Applicant: BEIJING MEIKANG GENO-IMMUNE BIOTECHNOLOGY CO., LTD., Beijing (CN)

(72) Inventors: Lung-Ji Chang, Beijing (CN); Yu-chen Liu, Beijing (CN)

(73) Assignee: BEIJING MEIKANG GENO-IMMUNE BIOTECHNOLOGY CO., LTD., Beijing (CN)

(21) Appl. No.: 18/850,948

(22) PCT Filed: Jun. 8, 2021
(86) PCT No.: PCT/CN2022/096639

§ 371 Date: Sep. 25, 2024

(30) Foreign Application Priority Data
Jun. 8, 2021 (CN) 202110640020.5
Mar. 18, 2022 (CN) 202210271824.7

(51) Int. Cl. A61K 35/17; A61K 40/11; A61K 40/31; A61K 40/42; A61P 35/02; C07K 14/705; C07K 14/725; C07K 16/28; C12N 5/0783; C12N 15/86

(52) U.S. Cl. CPC A61K 35/17; A61K 40/11; A61K 40/31; A61K 40/421; A61P 35/02; C07K 14/7051; C07K 14/70517; C07K 14/70521; C07K 14/70578; C07K 16/2803; C12N 5/0636; C12N 15/86; A61K 2239/13; A61K 2239/21; A61K 2239/48; C07K 2317/24; C07K 2317/622; C07K 2319/02; C07K 2319/03; C12N 2510/00; C12N 2740/15043

(57) ABSTRACT

The present application relates to a CD7-based humanized chimeric antigen receptor and use thereof, specifically a method for constructing chimeric antigen receptor T (CAR-T) cells based on a tumor-specific target CD7 and use thereof in anti-tumor therapy. The chimeric antigen receptor includes an antigen binding domain, a transmembrane domain, a costimulatory signaling region, and a CD3ζ signaling domain, which are connected in tandem; where the antigen binding domain binds to a tumor surface antigen, and the tumor surface antigen is CD7. The humanized chimeric antigen receptor of the present application performs particular gene modification on a single-chain antibody specific to an antigen CD7. The modified humanized single-chain antibody has a stronger antigen-antibody binding ability, and the chimeric antigen receptor stimulates T cells better, is maintained longer in vivo, and has a better targeting effect than other CD7 chimeric antigen receptors so that the therapeutic effect of CAR-T cells is enhanced.

### CROSS-REFERENCE TO RELATED APPLICATION(S)

This application claims priority to Chinese Patent Application No. 202110640020.5 (filed on Jun. 8, 2021 and titled CD7-BASED CHIMERIC ANTIGEN RECEPTOR AND USE THEREOF) and Chinese Patent Application No. 202210271824.7 (filed on Mar. 18, 2022 and titled CD7-BASED HUMANIZED CHIMERIC ANTIGEN RECEPTOR AND USE THEREOF), the disclosure of which is incorporated herein by reference in their entireties.

### TECHNICAL FIELD

The present application relates to the field of cellular immunotherapy of tumors and, in particular, to a CD7-based humanized chimeric antigen receptor and use thereof, specifically a method for constructing chimeric antigen receptor T (CAR-T) cells based on a tumor-specific target CD7 and use thereof in anti-tumor therapy.

### BACKGROUND

With the development of tumor immunology theories and clinical techniques, chimeric antigen receptor T (CAR-T) cell immunotherapy has become one of the most promising tumor immunotherapies. In general, a chimeric antigen receptor (CAR) consists of a tumor-associated antigen binding region, an extracellular hinge region, a transmembrane region, and an intracellular signal transduction region. Generally, the CAR contains a single-chain variable fragment (scFv) region of an antibody or a domain specifically binding to a tumor-associated antigen (TAA), which is coupled to a cytoplasmic domain of a T-cell signaling molecule through hinge and transmembrane regions. The most common lymphocyte activating moiety includes a T-cell costimulatory domain in tandem with a moiety (for example, CD3ζ) triggering the function of a T-cell effector. CAR-mediated adoptive immunotherapy allows CAR-transplanted T cells to directly recognize TAAs on target tumor cells in a non-HLA-restricted manner.

Most patients suffering from T-cell malignant tumors (including T-cell acute lymphocytic leukemia (T-ALL) and T-cell lymphoma) die of their diseases. One method for treating these patients is to perform gene modification on T cells through CAR expression to target antigens expressed on tumor cells. The CAR is an antigen receptor designed to recognize cell surface antigens in a human leukocyte antigen (HLA)-independent manner. An attempt to treat these types of patients with CAR-expressing genetically modified cells has made a promising success.

CD19 molecules are potential targets for the treatment of B lymphocytic tumors and also a hotspot in CAR-related research. The expression of CD19 is limited to normal and malignant B cells, and CD19 is a widely accepted CAR target. Chimeric antigen receptor genetically modified T cells targeting CD19 molecules (CD19 CAR-T) have achieved a great success in treating resistant and refractory B cell acute lymphocytic leukemia. However, related CAR-T treatment techniques for treating refractory and recurrent T lymphocytic leukemia and T lymphocytic lymphoma are relatively slow and lacking in development.

CN104788573A discloses a chimeric antigen receptor hCD19scFv-CD8α-CD28-CD3ζ and a use thereof. The chimeric antigen receptor consists of light chain and heavy chain variable regions of an anti-human CD19 monoclonal antibody HI19a (hCD19scFv), a hinge region of human CD8α, a transmembrane region and an intracellular region of human CD28, and an intracellular region of human CD3ζ, which are connected in tandem. In this patent, the expression of CD19 is reduced after one CAR-T cell infusion but a single target immune mechanism is easy to escape by the tumor cells. However, the application of CAR-T technology for targeting T cell tumors is relatively lacking.

The CD7 molecule is a cell surface glycoprotein having a molecular weight of 40×10³, belongs to an immunoglobulin superfamily, and plays an important role in lymphocyte maturation. When CD7 molecules adhere to an antibody or an antibody derivative, endocytosis occurs rapidly. This property makes CD7 very appropriate to be targeted by immunotoxins, where the immunotoxins along with antigens are internalized and then poison cells from the inside of the cells. However, CD7 remains unknown as a cell surface targeting molecule of the CAR. Whether CD7 CAR-T cells can effectively target T-cell tumor still requires to be confirmed through experiments. Studies have shown that CD7 is highly expressed in many types of T-ALL and T-cell lymphoma so that CD7 is likely to become one of the potential therapeutic targets for T lymphoid hematopoietic tumors.

The current CAR-T technology targeting the T-cell tumors is not well established yet, and a tumor microenvironment that affects the therapeutic effect of CAR-T is still a challenge. Therefore, a CD7-based chimeric antigen receptor (CD7 CAR-T) technology has been developed, which has a great potential for the treatment of recurrent/refractory CD7-positive T-cell malignant tumors.

### SUMMARY

The present application provides a CD7-based humanized chimeric antigen receptor and use thereof. The chimeric antigen receptor prepared in the present application improves the immune effect of a target and enhances the therapeutic effect of CAR-T cells through CD7-targeted gene modification of T cells so that CD7 CAR-T cells are expected to become a new direction for the treatment of T-cell tumor.

In a first aspect, the present application provides a CD7-based humanized chimeric antigen receptor. The humanized chimeric antigen receptor includes an antigen binding domain, a transmembrane domain, a costimulatory signaling region, and a CD3ζ signaling domain, which are connected in tandem; wherein the antigen binding domain binds to a tumor surface antigen, and the tumor surface antigen is CD7.

In the present application, the antigen binding domain binds to the tumor surface antigen CD7, and then particular humanized gene modification is performed on the CD7 chimeric antigen receptor so that the tumor surface antigen CD7 can specifically bind to the chimeric antigen receptor of the present application. Compared with other chimeric antigen receptors and other tumor antigens, the chimeric antigen receptor has an improved effect and can be expressed at high levels to the targets that the immune effect of CAR-T cells is enhanced.

Preferably, the antigen binding domain includes a humanized CD7 single-chain antibody.

Preferably, a nucleotide sequence of the humanized CD7 single-chain antibody includes a sequence having more than 80% homology to a sequence shown in SEQ ID NO. 1.

Preferably, an amino acid sequence of the humanized CD7 single-chain antibody includes a sequence having more than 80% homology to a sequence shown in SEQ ID NO. 2.

Preferably, a nucleotide sequence of the humanized chimeric antigen receptor includes a sequence having more than 80% homology to a sequence shown in SEQ ID NO. 3, SEQ ID NO. 4, or SEQ ID NO. 5.

Preferably, an amino acid sequence of the humanized chimeric antigen receptor includes a sequence having more than 80% homology to a sequence shown in SEQ ID NO. 6, SEQ ID NO. 7, or SEQ ID NO. 8.

SEQ ID NO. 1:
gaagtccaacttgttgaatcaggaggtggcttggtaaaacctgggggctccctcaaacttagctgcgcggcatccggcctcactttta
gctcttacgcaatgtcatgggtgagacagacaccggaaaagcgccttgagtgggttgcaagcatatcaagtggtgggttcacatactatcca
gacagtgtaaaagggcggttcacgatttcaagagacaacgcaagaaacatactgtacctgcagatgagctctctccgatccgaagatacag
ctatgtattactgcgcgagagatgaggtacgcggttaccttgatgtctggggagcggggactacagtgacagtctccagtggctccacttcc
ggtagcggcaagcctggtagttccgagggcagtaccaagggggacattcagatgacgcagacgacttcctcactttcagccagtcttggtg
atcgcgtcactataagttgttctgcatcacaaggtatatctaattacctgaattggtatcaacaaaagcccgacggcactgtcaagttgttga
tatattacacctcctcactccattctggggtcccgtcccgattcagtggtagcgggtctggaacggactactccctcaccatttcaaacttgg
agcccgaggatattgctacctactattgtcagcagtactcaaaactgccttacacctttggagggggcacaaaactcgagataaaa.
SEQ ID NO. 2:
EVQLVESGGGLVKPGGSLKLSCAASGLTFSSYAMSWVRQTPEKRLEWVASISSGGFT
YYPDSVKGRFTISRDNARNILYLQMSSLRSEDTAMYYCARDEVRGYLDVWGAGTTVTV
SSGSTSGSGKPGSSEGSTKGDIQMTQTTSSLSASLGDRVTISCSASQGISNYLNWYQQKP
DGTVKLLIYYTSSLHSGVPSRFSGSGSGTDYSLTISNLEPEDIATYYCQQYSKLPYTFGGG
TKLEIK.

In the present application, the humanized CD7 single-chain antibody (CD7 scFv) is obtained through humanized engineering and specific modification so that an antibody expressed by the modified sequence has a stronger antigen-antibody binding ability and can be expressed in vivo more stably for a long term.

Preferably, the amino acid sequence of the humanized CD7 single-chain antibody includes a sequence having more than 90% homology to the sequence shown in SEQ ID NO. 2.

Preferably, the nucleotide sequence of the humanized chimeric antigen receptor includes a sequence having more than 80% homology to the sequence shown in SEQ ID NO. 3, SEQ ID NO. 4, or SEQ ID NO. 5.

Preferably, the amino acid sequence of the humanized chimeric antigen receptor includes a sequence having more than 80% homology to the sequence shown in SEQ ID NO. 6, SEQ ID NO. 7, or SEQ ID NO. 8.

Preferably, the transmembrane domain is a CD28 transmembrane domain and/or a CD8α transmembrane domain. In some embodiments, the transmembrane domain may be selected or modified through an amino acid substitution.

Preferably, the costimulatory signaling region is a combination of a CD28 signaling domain and a 4-1BB, CD27, or IL-15R signaling domain. The arrangement of the CD28 signaling domain and the 4-1BB, CD27, or IL-15R signaling domain may be adjusted by those skilled in the art as required, and different arrangements of the CD28 signaling domain and the 4-1BB, CD27, or IL-15R signaling domain have no effect on the humanized chimeric antigen receptor.

Preferably, the humanized chimeric antigen receptor further includes a self-destructing domain, and the self-destructing domain includes a caspase 9 domain.

Preferably, the self-destructing domain is connected in tandem with the CD3ζ signaling domain through a 2A sequence. The 2A sequence makes a protein expressed by the self-destructing domain be broken from a protein of the humanized chimeric antigen receptor so that the humanized chimeric antigen receptor can exert its effect. An activator is injected to activate the self-destructing domain so that the humanized chimeric antigen receptor loses its effect.

Preferably, the humanized chimeric antigen receptor further includes a signal peptide. The signal peptide may be any signal peptide capable of directing the transmembrane transfer of the chimeric antigen receptor, and those skilled in the art may select a conventional signal peptide in the art as required.

Preferably, the signal peptide is a Secretory signal peptide, and the Secretory signal peptide may be a signal peptide of a CD8a gene, which has a sequence of 21 amino acids: MALPVTALLLPLALLLHAARP (SEQ ID NO. 11), or a signal peptide of a GM-CSFR gene, which has a sequence of 22 amino acids: MLLLVTSLLLCELPHPAFLLIP (SEQ ID NO. 12), or a signal peptide of any secretory protein gene.

The humanized chimeric antigen receptor of the present application may further include a hinge region, such as GGGGS (SEQ ID NO. 13) or GGGGSGGGGS (SEQ ID NO. 14). The hinge region may be selected by those skilled in the art according to actual situations and is not specially limited herein. The presence of the hinge region does not affect the performance of the humanized chimeric antigen receptor of the present application.

Preferably, the humanized chimeric antigen receptor includes a signal peptide, an antigen binding domain, a transmembrane domain, a costimulatory signaling region, a CD3ζ signaling domain, a 2A sequence, and a self-destructing domain, which are connected in tandem.

As a preferred technical solution, the humanized chimeric antigen receptor includes a Secretory signal peptide, a humanized CD7 single-chain antibody (CD7 scFv), a CD8α transmembrane domain and/or a CD28 transmembrane domain, a combination of a CD28 signaling domain and a 4-1BB, CD27, or IL-15R signaling domain, a CD3ζ signaling domain, a 2A sequence, and a caspase 9 domain, which are connected in tandem. Specifically, they may be arranged as the following combinations:
Secretory signal peptide-humanized CD7 single-chain antibody-CD28-4-1BB(CD137)-CD3ζ; or
Secretory signal peptide-humanized CD7 single-chain antibody-CD28-CD27-CD3ζ-2A-iCasp9; or
Secretory signal peptide-humanized CD7 single-chain antibody-CD28-IL-15R-CD3ζ.

Preferably, the open reading frame (ORF) of the gene of Secretory signal peptide-humanized CD7 single-chain antibody-CD28-4-1BB-CD3ζ-2A-iCasp9 has a nucleotide sequence as shown in SEQ ID NO. 3.

SEQ ID NO. 3:
atgctcctgcttgtaacgagcctcctgctctgtgagctgcctcaccccgctttccttcttataccggaagtccaacttgttgaatcaggag
gtggcttggtaaaacctgggggctccctcaaacttagctgcgcggcatccggcctcacttttagctcttacgcaatgtcatgggtgagacaga
caccggaaaagcgccttgagtgggttgcaagcatatcaagtggtgggttcacatactatccagacagtgtaaaagggcggttcacgatttca
agagacaacgcaagaaacatactgtacctgcagatgagctctctccgatccgaagatacagctatgtattactgcgcgagagatgaggtac
gcggttaccttgatgtctggggagcggggactacagtgacagtctccagtggctccacttccggtagcggcaagcctggtagttccgaggg
cagtaccaagggggacattcagatgacgcagacgacttcctcactttcagccagtcttggtgatcgcgtcactataagttgttctgcatcaca
aggtatatctaattacctgaattggtatcaacaaaagcccgacggcactgtcaagttgttgatatattacacctcctcactccattctggggt
cccgtcccgattcagtggtagcgggtctggaacggactactccctcaccatttcaaacttggagcccgaggatattgctacctactattgtca
gcagtactcaaaactgccttacacctttggagggggcacaaaactcgagataaaagcggccgcaattgaagttatgtatcctcctccttacct
agacaatgagaagagcaatggaaccattatccatgtgaaagggaaacacctttgtccaagtcccctatttcccggaccttctaagcccttttg
ggtgctggtggtggttgggggagtcctggcttgctatagcttgctagtaacagtggcctttattattttctgggtgaggagtaagaggagcag
gctcctgcacagtgactacatgaacatgactccccgccgccctgggcccacccgcaagcattaccagccctatgccccaccacgcgacttcgc
agcctatcgctccgctagcggcggaggtggctctggcggtggcggatcagttgttaaacggggcagaaagaaactcctgtatatattcaaaca
accatttatgagaccagtacaaactactcaagaggaagatggctgtagctgccgatttccagaagaagaagaaggaggatgtgaactgggcg
gcggcggcagcggaggcggtggcagcggcggcggcggctctagagtgaagttcagcaggagcgcagacgcccccgcgtaccagca
gggccagaaccagctctataacgagctcaatctaggacgaagagaggagtacgatgttttggacaagagacgtggccgggaccctgaga
tggggggaaagccgagaaggaagaaccctcaggaaggcctgtacaatgaactgcagaaagataagatggcggaggcctacagtgagat
tgggatgaaaggcgagcgccggaggggcaaggggcacgatggcctttaccagggtctcagtacagccaccaaggacacctacgacgc
ccttcacatgcaggccctgccccctcgcactagttaa.

Preferably, the ORF of the gene of the humanized chimeric antigen receptor: Secretory signal peptide-humanized CD7 single-chain antibody-CD28-CD27-CD3ζ-2A-iCasp9 has a nucleotide sequence as shown in SEQ ID NO. 4.

SEQ ID NO. 4:
atgctcctgcttgtaacgagcctcctgctctgtgagctgcctcaccccgctttccttcttataccggaagtccaacttgttgaatcaggag
gtggcttggtaaaacctgggggctccctcaaacttagctgcgcggcatccggcctcacttttagctcttacgcaatgtcatgggtgagacaga
caccggaaaagcgccttgagtgggttgcaagcatatcaagtggtgggttcacatactatccagacagtgtaaaagggcggttcacgatttca
agagacaacgcaagaaacatactgtacctgcagatgagctctctccgatccgaagatacagctatgtattactgcgcgagagatgaggtac
gcggttaccttgatgtctggggagcggggactacagtgacagtctccagtggctccacttccggtagcggcaagcctggtagttccgaggg
cagtaccaagggggacattcagatgacgcagacgacttcctcactttcagccagtcttggtgatcgcgtcactataagttgttctgcatcac
aaggtatatctaattacctgaattggtatcaacaaaagcccgacggcactgtcaagttgttgatatattacacctcctcactccattctggg
gtcccgtcccgattcagtggtagcgggtctggaacggactactccctcaccatttcaaacttggagcccgaggatattgctacctactattg
tcagcagtactcaaaactgccttacacctttggagggggcacaaaactcgagataaaagcggccgcaattgaagttatgtatcctcctcctt
acctagacaatgagaagagcaatggaaccattatccatgtgaaagggaaacacctttgtccaagtcccctatttcccggaccttctaagccc
ttttgggtgctggtggtggttgggggagtcctggcttgctatagcttgctagtaacagtggcctttattattttctgggtgaggagtaagag
gagcaggctcctgcacagtgactacatgaacatgactccccgccgccctgggcccacccgcaagcattaccagccctatgccccaccacgcg
acttcgcagcctatcgctccgctagcggaggtggaggttctggaggtggtggaagtcaaagaaggaagtaccgcagcaacaaaggagaatct
cccgtcgagccagccgagccctgtcattattcatgcccaagggaggaggagggaagtacaatcccaattcaagaagactacaggaagcccga
acctgcatgcagtccaggtggaggcggttctggaggcggtggctcccgggtgaaattctcacggtctgcagacgcacccgcttaccagcaag
gccagaaccaactctataacgagctcaatctaggacgaagagaggagtacgatgttttggacaagagacgtggccgggaccctgagatggg
gggaaagccgagaaggaagaaccctcaggaaggcctgtacaatgaactgcagaaagataagatggcggaggcctacagtgagattggg
atgaaaggcgagcgccggaggggcaaggggcacgatggcctttaccagggtctcagtacagccaccaaggacacctacgacgcccttc
acatgcaggccctgccccctcgcactagtggctccggagccacgaacttctctctgttaaagcaagcaggagacgtggaagaaaaccccg
gtcccatgggagtgcaggtggaaaccatctccccaggagacgggcgcaccttccccaagcgcggccagacctgcgtggtgcactacac
cgggatgcttgaagatggaaagaaagtggactcctcccgggacagaaacaagccctttaagtttatgctaggcaagcaggaggtgatccg
aggctgggaagaaggggttgcccagatgagtgtgggtcagagagccaaactgactatatctccagattatgcctatggtgccactgggcac
ccaggcatcatcccaccacatgccactctcgtcttcgatgtggagcttctaaaactggaaggtggaggcggttcaggcggcggcggcagc
ggcgccatggtcggtgctcttgagagtttgaggggaaatgcagatttggcttacatcctgagcatggagccctgtggccactgcctcattat
caacaatgtgaacttctgccgtgagtccgggctccgcacccgcactggctccaacatcgactgtgagaagttgcggcgtcgcttctcctcgc
tgcatttcatggtggaggtgaagggcgacctgactgccaagaaaatggtgctggctttgctggagctggcgcggcaggaccacggtgctct
ggactgctgcgtggtggtcattctctctcacggctgtcaggccagccacctgcagttcccaggggctgtctacggcacagatggatgccct
gtgtcggtcgagaagattgtgaacatcttcaatgggaccagctgccccagcctgggagggaagcccaagctctttttcatccaggcctgtgg
tggggagcagaaagaccatgggtttgaggtggcctccacttcccctgaagacgagtcccctggcagtaaccccgagccagatgccaccc
cgttccaggaaggtttgaggaccttcgaccagctggacgccatatctagtttgcccacacccagtgacatctttgtgtcctactctactttc
ccaggttttgtttcctggagggaccccaagagtggctcctggtacgttgagaccctggacgacatctttgagcagtgggctcactctgaaga
cctgcagtccctcctgcttagggtcgctaatgctgtttcggtgaaagggatttataaacagatgcctggttgctttaatttcctccggaaaa
aacttttctttaaaacatcagctagttaa.

Preferably, the ORF of the gene of Secretory signal peptide-humanized CD7 single-chain antibody-CD28-IL-15R-CD3ζ has a nucleotide sequence as shown in SEQ ID NO. 5.

SEQ ID NO. 5:
atgctcctgcttgtaacgagcctcctgctctgtgagctgcctcaccccgctttccttcttataccggaagtccaacttgttgaatcaggag
gtggcttggtaaaacctgggggctccctcaaacttagctgcgcggcatccggcctcacttttagctcttacgcaatgtcatgggtgagacag
acaccggaaaagcgccttgagtgggttgcaagcatatcaagtgggggttcacatactatccagacagtgtaaaagggcggttcacgatttca
agagacaacgcaagaaacatactgtacctgcagatgagctctctccgatccgaagatacagctatgtattactgcgcgagagatgaggtac
gcggttaccttgatgtctggggagcggggactacagtgacagtctccagtggctccacttccggtagcggcaagcctggtagttccgaggg
cagtaccaagggggacattcagatgacgcagacgacttcctcactttcagccagtcttggtgatcgcgtcactataagttgttctgcatcac
ggtatatctaattacctgaattggtatcaacaaaagcccgacggcactgtcaagttgttgatatattacacctcctcactccattctggggt
cccaagtcccgattcagtggtagcgggtctggaacggactactccctcaccatttcaaacttggagcccgaggatattgctacctactattg
tcagcagtactcaaaactgccttacacctttggagggggcacaaaactcgagataaaagcggccgcaattgaagttatgtatcctcctcctt
acctagacaatgagaagagcaatggaaccattatccatgtgaaagggaaacacctttgtccaagtcccctatttcccggaccttctaagccc
ttttgggtgctggtggtggttgggggagtcctggcttgctatagcttgctagtaacagtggcctttattattttctgggtgaggagtaagag
gagcaggctcctgcacagtgactacatgaacatgactccccgccgccctgggcccacccgcaagcattaccagccctatgccccaccacgcg
acttcgcagcctatcgctccgctagcggtggaggtggatctggtggtggaggttccaaatctcggcagacaccacctctcgcatctgtcgag
atggaggccatggaggcactgcctgtcacatggggtacttccagcagggacgaggacctcgaaaattgctcccatcatctgggtggcggcgg
ttccggtggtggaggttctcgggtgaaattctccaggtccgcagacgctccagcctaccagcaaggacagaaccaactgtacaatgagctga
atctgggaagacgcgaggagtatgacgtgctggacaaacggagaggtcgcgaccccgagatgggaggcaagcctcgcaggaagaacccacag
gaaggactgtacaacgaactgcagaaggacaagatggccgaggcatactcagagatcggaatgaagggcgagagacgcagagggaa
aggccacgacggactgtaccagggactgtcaactgccacaaaggatacttacgatgccctgcacatgcaggcactccctccacgctga.

Preferably, the gene of Secretory signal peptide-humanized CD7 single-chain antibody-CD28-4-1BB-CD3ζ-2A-iCasp9 has an amino acid sequence as shown in SEQ ID NO. 6.

SEQ ID NO. 6:
MLLLVTSLLLCELPHPAFLLIPEVQLVESGGGLVKPGGSLKLSCAASGLTFSSYAMSW
VRQTPEKRLEWVASISSGGFTYYPDSVKGRFTISRDNARNILYLQMSSLRSEDTAMYYCA
RDEVRGYLDVWGAGTTVTVSSGSTSGSGKPGSSEGSTKGDIQMTQTTSSLSASLGDRVT
ISCSASQGISNYLNWYQQKPDGTVKLLIYYTSSLHSGVPSRFSGSGSGTDYSLTISNLEPE
DIATYYCQQYSKLPYTFGGGTKLEIKAAAIEVMYPPPYLDNEKSNGTIIHVKGKHLCPSP
LFPGPSKPFWVLVVVGGVLACYSLLVTVAFIIFWVRSKRSRLLHSDYMNMTPRRPGPTR
KHYQPYAPPRDFAAYRSASGGGGSGGGGSVVKRGRKKLLYIFKQPFMRPVQTTQEEDG
CSCRFPEEEEGGCELGGGGSGGGGSGGGGSRVKFSRSADAPAYQQGQNQLYNELNLGR
REEYDVLDKRRGRDPEMGGKPRRKNPQEGLYNELQKDKMAEAYSEIGMKGERRRGKG
HDGLYQGLSTATKDTYDALHMQALPPRTS.

Preferably, the gene of Secretory signal peptide-humanized CD7 single-chain antibody-CD28-CD27-CD3ζ-2A-iCasp9 has an amino acid sequence as shown in SEQ ID NO. 7.

SEQ ID NO. 7:
MLLLVTSLLLCELPHPAFLLIPEVQLVESGGGLVKPGGSLKLSCAASGLTFSSYAMSW
VRQTPEKRLEWVASISSGGFTYYPDSVKGRFTISRDNARNILYLQMSSLRSEDTAMYYCA
RDEVRGYLDVWGAGTTVTVSSGSTSGSGKPGSSEGSTKGDIQMTQTTSSLSASLGDRVT
ISCSASQGISNYLNWYQQKPDGTVKLLIYYTSSLHSGVPSRFSGSGSGTDYSLTISNLEPE
DIATYYCQQYSKLPYTFGGGTKLEIKAAAIEVMYPPPYLDNEKSNGTIIHVKGKHLCPSP
LFPGPSKPFWVLVVVGGVLACYSLLVTVAFIIFWVRSKRSRLLHSDYMNMTPRRPGPTR
KHYQPYAPPRDFAAYRSASGGGGSGGGGSQRRKYRSNKGESPVEPAEPCHYSCPREEEG
STIPIQEDYRKPEPACSPGGGGSGGGGSRVKFSRSADAPAYQQGQNQLYNELNLGRREEY
DVLDKRRGRDPEMGGKPRRKNPQEGLYNELQKDKMAEAYSEIGMKGERRRGKGHDGL
YQGLSTATKDTYDALHMQALPPRTSGSGATNFSLLKQAGDVEENPGPMGVQVETISPGD
GRTFPKRGQTCVVHYTGMLEDGKKVDSSRDRNKPFKFMLGKQEVIRGWEEGVAQMSV
GQRAKLTISPDYAYGATGHPGIIPPHATLVFDVELLKLEGGGGSGGGGSGAMVGALESLR
GNADLAYILSMEPCGHCLIINNVNFCRESGLRTRTGSNIDCEKLRRRFSSLHFMVEVKGD
LTAKKMVLALLELARQDHGALDCCVVVILSHGCQASHLQFPGAVYGTDGCPVSVEKIV
NIFNGTSCPSLGGKPKLFFIQACGGEQKDHGFEVASTSPEDESPGSNPEPDATPFQEGLRTF
DQLDAISSLPTPSDIFVSYSTFPGFVSWRDPKSGSWYVETLDDIFEQWAHSEDLQSLLLRV
ANAVSVKGIYKQMPGCFNFLRKKLFFKTSAS.

Preferably, the gene of Secretory signal peptide-humanized CD7 single-chain antibody-CD28-IL-15Ra-CD3ζ has an amino acid sequence as shown in SEQ ID NO. 8.

SEQ ID NO. 8:
MLLLVTSLLLCELPHPAFLLIPEVQLVESGGGLVKPGGSLKLSCAASGLTFSSYAMSW
VRQTPEKRLEWVASISSGGFTYYPDSVKGRFTISRDNARNILYLQMSSLRSEDTAMYYCA
RDEVRGYLDVWGAGTTVTVSSGSTSGSGKPGSSEGSTKGDIQMTQTTSSLSASLGDRVT
ISCSASQGISNYLNWYQQKPDGTVKLLIYYTSSLHSGVPSRFSGSGSGTDYSLTISNLEPE
DIATYYCQQYSKLPYTFGGGTKLEIKAAAIEVMYPPPYLDNEKSNGTIIHVKGKHLCPSP
LFPGPSKPFWVLVVVGGVLACYSLLVTVAFIIFWVRSKRSRLLHSDYMNMTPRRPGPTR
KHYQPYAPPRDFAAYRSASGGGGSGGGGSKSRQTPPLASVEMEAMEALPVTWGTSSRD
EDLENCSHHLGGGGSGGGGSRVKFSRSADAPAYQQGQNQLYNELNLGRREEYDVLDKR
RGRDPEMGGKPRRKNPQEGLYNELQKDKMAEAYSEIGMKGERRRGKGHDGLYQGLST
ATKDTYDALHMQALPPR.

Preferably, a method for preparing the humanized chimeric antigen receptor includes: transducing a nucleotide sequence encoding the humanized chimeric antigen receptor into a T cell for expression.

Preferably, the nucleotide sequence is transduced into the T cell through any one or a combination of at least two of a viral vector, an eukaryotic expression plasmid, or an mRNA sequence; preferably, the nucleotide sequence is transduced into the T cell through the viral vector.

Preferably, the viral vector is any one or a combination of at least two of a lentiviral vector or a retroviral vector, preferably a lentiviral vector.

Preferably, the vector expressing the humanized chimeric antigen receptor further includes a promoter which is any one or a combination of at least two of EF1a, CMV-TAR, or CMV.

In a second aspect, the present application provides a recombinant lentivirus. The recombinant lentivirus includes a vector expressing the CD7-based humanized chimeric antigen receptor described in the first aspect.

Preferably, a method for preparing the recombinant lentivirus includes co-transfecting a viral vector expressing the CD7-based humanized chimeric antigen receptor described in the first aspect and packaging helper plasmids pNHP and pHEF-VSVG into a mammalian cell obtain the recombinant lentivirus.

Preferably, the mammalian cell is any one or a combination of at least two of 293 cells, 293T cells, or TE671 cells.

In a third aspect, the present application provides a composition. The composition includes the CD7-based humanized chimeric antigen receptor described in the first aspect and/or the recombinant lentivirus described in the second aspect.

In a fourth aspect, the present application provides use of the CD7-based humanized chimeric antigen receptor described in the first aspect, the recombinant lentivirus described in the second aspect, or the composition described in the third aspect to preparation of a chimeric antigen receptor T cell or a medicament for treating a tumor.

Preferably, the tumor is a blood-related tumor disease, and the blood-related tumor disease is T-cell-associated leukemia or lymphoma.

Compared with the existing art, the present application has the beneficial effects described below.
(1) The humanized chimeric antigen receptor of the present application is obtained through particular gene modification on a CD7 antibody, and the humanized antibody after modification can have a stronger surface antigen-antibody binding ability.
(2) The humanized CD7 chimeric antigen receptor of the present application can specifically recognize the tumor surface antigen CD7. CD7 is highly expressed in the T-cell-associated leukemia and lymphoma. Compared with other chimeric antigen receptors, the humanized CD7 chimeric antigen receptor has the better effect so that the immune effect of CAR-T cells is enhanced, an in vivo survival time is prolonged, and the therapeutic effect of the CAR-T cells is enhanced.
(3) After CAR-T cell infusion, the chimeric antigen receptor of the present application effectively targets CD7 on the surface of the tumor so that the tumor cannot easily escape an immune mechanism and can be better treated.

### BRIEF DESCRIPTION OF DRAWINGS

FIG. 1 is a schematic diagram of a safe and effective application of lentiviral vector-targeted CAR-T;

FIG. 2 is a schematic map of a synthetic genetic structure of a chimeric antigen receptor of the present application;

FIG. 3 shows the experimental results of two different CD7 CAR-T cells killing tumor cell strains in vitro;

FIG. 4A is a flow chart illustration of a clinical trial of CD7 CAR-T;

FIG. 4B shows the expression results of CD7 in the bone marrow of patients with T-ALL;

FIG. 4C shows the expression result of CD7 on a tumor specimen fixed section of a patient with T lymphoblastoma; and

FIG. 4D shows the kinetic curves of the detected CAR copy numbers in the blood cells in patients after CD7 CAR-T infusion.

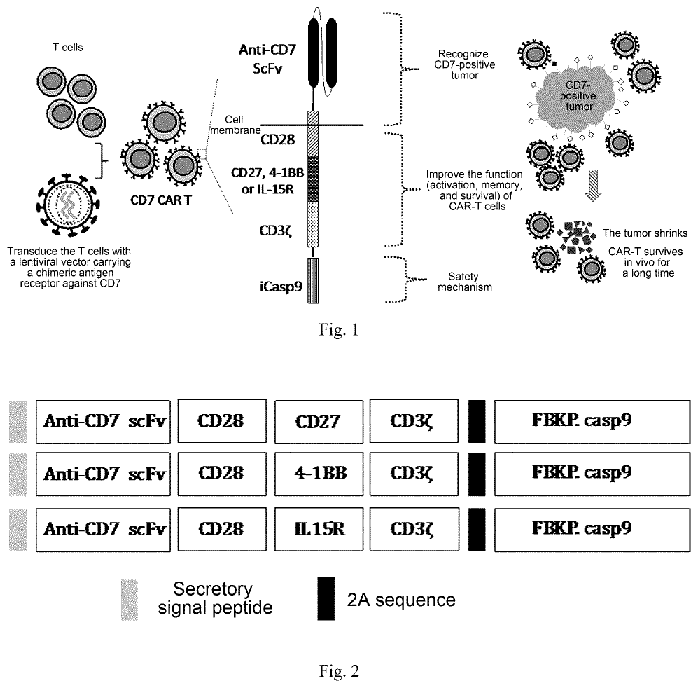

### DETAILED DESCRIPTION

To further elaborate on the technical means adopted and the effects achieved by the present application, solutions of the present application are further described below through specific examples in conjunction with drawings, but the present application is not limited to the scope of the examples.

Experiments without specific techniques or conditions noted in the examples are conducted according to techniques or conditions described in the literature in the art or a product specification. The reagents or instruments used herein without manufacturers specified are conventional products commercially available from proper channels.

### Example 1. Construction of Chimeric Antigen Receptors

Nucleotide sequences encoding chimeric antigen receptors were obtained through gene synthesis, where the chimeric antigen receptors included a Secretory signal peptide, an antigen binding domain CD7 scFv, CD8a and/or CD28 transmembrane domains, a combination of a CD28 signaling domain and a 4-1BB, CD27, or IL-15R signaling domain, a CD3ζ signaling domain, a 2A sequence, and a caspase 9 domain. The nucleotide sequences of chimeric antigen receptors are shown in SEQ ID NO. 3, SEQ ID NO. 4, and SEQ ID NO. 5, respectively. The nucleotide sequences of the three chimeric antigen receptors were inserted into pTYF lentiviral vectors, separately.

### Example 2 Lentivirus Packaging

(1) 293T cells were cultured for 18 h.
(2) Fresh DMEM containing 10% fetal bovine serum (FBS) was added.
(3) DMEM supernatant was taken from each well, added in a sterile centrifuge tube together with the following reagents: DNA mix (pNHP and pHEF-VSV-G) and pTYF DNA vectors, and vortexed.
(4) Superfect was aspirated, added to the centrifuge tube, and allowed to stand at 25° C. for 8 min.
(5) The DNA-Superfect mixture in the centrifuge tube was added dropwise to cultured cells and mixed.
(6) The system was incubated at 37° C. in a 3% CO₂ incubator for 4 h.
(7) The solution in the culture medium was aspirated, added with AIM-V, and continued to be cultured.
(8) The cultured cells were placed back to the CO₂ incubator and cultured overnight. The transduction efficiency was observed the next morning with a microscope.

### Example 3 Purification and Concentration of Lentiviruses

### (1) Purification of Viral Vectors

Cell debris was removed through centrifugation to obtain the virus supernatant, the virus supernatant was filtered by a low protein binding filter, and the viruses were divided into small portions and stored at −80° C.

Generally, the transduced cells may produce lentiviral vectors with a titer of greater than 10⁷ transduction units per milliliter of the culture medium.

(2) Concentration of Lentiviral Vectors with Centricon or a Similar Filter
1) Centricon tubes were taken from a biosafety cabinet, disinfected with alcohol, and washed with sterile PBS.
2) Virus supernatant was added to each Centricon filter and centrifuged for 30 min or centrifuged until the virus volume was reduced to 0.5 mL.
3) The filter tube was shaken and centrifuged for 2 min, the concentrated viruses were collected into a collection cup, and finally the viruses in all tubes were concentrated into one centrifuge tube to obtain the lentiviruses expressing the three chimeric antigen receptors separately.

### Example 4 Lentiviral Transduction and Preparation of CAR-T Cells

The activated T cells were inoculated and suspended in a culture medium and added with 10 μg/mL of polybrene. The culture medium was AIM-V containing cell culture factors IL-2, IL-7, and IL-15. Three CAR gene lentiviruses concentrated and prepared in Example 3 were added, centrifuged for 100 min at 25° C. at a speed of a 100×g centrifugal force, and cultured for 24 h at 37° C. A culture medium was added and cultured for 4 days, the cells were harvested and counted, and the supernatant was tested for endotoxin and mycoplasma. The cells were amplified in vitro for two days and then infused to patients.

### Example 5 In Vitro Tumor Killing of CAR-T Cells

Green fluorescent protein genes were transferred into a CD7-positive human T lymphoma cell strain (Molt-3) via lentiviral vectors and stably expressed as target cells.

Unmodified T cells and non-specific GD2 CAR-T cells were used as negative control groups, and five types of CD7 CAR-T were used as experimental groups, where:
two types of CD7 CAR-T with scFVs in series A including signaling domains CD28-CD27-CD3ζ and CD28-41BB-CD3ζ respectively and were abbreviated as A-CD27 (with a nucleotide sequence SEQ ID NO. 9) and A-41BB (with a nucleotide sequence SEQ ID NO. 10) respectively, and
the other three types of CD7 CAR-T were humanized CD7 CAR-T with scFVs in series H which were optimized through the design of the present application, and included signaling domains CD28-CD27-CD3ζ, CD28-41BB-CD3ζ, and CD28-IL15R-CD3ζ and were abbreviated as H-CD27 (SEQ ID NO. 4), H-41BB (SEQ ID NO. 3), and H-IL15R (SEQ ID NO. 5) respectively.

The above CAR-T and Molt-3 tumor were incubated at a ratio of 2:1 in a 37° C. and 5% CO₂ incubator for 24 h.

SEQ ID NO. 9:
atgctcctgcttgtgacctcactcttgctttgtgaactgccacatccggcctttctgttgatcccgcaagtccaacttgttgagtcaggag
gcggtctcgtgcagccaggtggatcacgcaaactttcttgtgcagcttccggattcacattcagtagtttcgggatgcattgggtcaggcaag
ctccagagaaagggctcgaatgggtagcgtatatctctagtggatctagtacgctgcattacgctgacaccgtcaaaggtcggtttacgatta
gcagggataaccccaagaatacgctgttcttgcagatgacatcattgcggtctgaagatacggccatgtactactgcgcccggtgggggaa
ctatccccattatgcgatggattactggggtcaaggcacctctgtcacggtttcttccggaagcacgagtgggagtgggaaacctggcagct
ctgaggggagtacgaaaggtgatatagtgatgacacaaagccccgcctcacttgcagtgtctcttggggacagagcgaccatcagttgccg
agccagcaaatctgtttctgcctctggatacagttatatgcactggtatcaacagaaaccggggcaaccgccaaagttgctcatatacttggc
gtcaaatctcgaatccggtgttcctgcacggttctccggctctggatctggtacagatttcactctcaacatccatcccgtcgaggaggagga
cgctgtaacctactactgtcagcattccagggagcttccttacacatttggcggcggcacaaagctcgagattaaggcggccgcaattgaag
ttatgtatcctcctccttacctagacaatgagaagagcaatggaaccattatccatgtgaaagggaaacacctttgtccaagtcccctatttc
ccggaccttctaagcccttttgggtgctggtggtggttgggggagtcctggcttgctatagcttgctagtaacagtggcctttattattttct
gggtgaggagtaagaggagcaggctcctgcacagtgactacatgaacatgactccccgccgccctgggcccacccgcaagcattaccagccct
atgccccaccacgcgacttcgcagcctatcgctccgctagcggaggtggaggttctggaggtggtggaagtcaaagaaggaagtaccgca
gcaacaaaggagaatctcccgtcgagccagccgagccctgtcattattcatgcccaagggaggaggagggaagtacaatcccaattcaag
aagactacaggaagcccgaacctgcatgcagtccaggtggaggcggttctggaggcggtggctcccgggtgaaattctcacggtctgca
gacgcacccgcttaccagcaaggccagaaccaactctataacgagctcaatctaggacgaagagaggagtacgatgttttggacaagaga
cgtggccgggaccctgagatggggggaaagccgagaaggaagaaccctcaggaaggcctgtacaatgaactgcagaaagataagatg
gcggaggcctacagtgagattgggatgaaaggcgagcgccggaggggcaaggggcacgatggcctttaccagggtctcagtacagcc
accaaggacacctacgacgcccttcacatgcaggccctgccccctcgcactagt.
SEQ ID NO. 10:
atgctcctgcttgtgacctcactcttgctttgtgaactgccacatccggcctttctgttgatcccgcaagtccaacttgttgagtcaggag
gcggtctcgtgcagccaggtggatcacgcaaactttcttgtgcagcttccggattcacattcagtagtttcgggatgcattgggtcaggcaag
ctccagagaaagggctcgaatgggtagcgtatatctctagtggatctagtacgctgcattacgctgacaccgtcaaaggtcggtttacgatta
gcagggataaccccaagaatacgctgttcttgcagatgacatcattgcggtctgaagatacggccatgtactactgcgcccggtgggggaa
ctatccccattatgcgatggattactggggtcaaggcacctctgtcacggtttcttccggaagcacgagtgggagtgggaaacctggcagct
ctgaggggagtacgaaaggtgatatagtgatgacacaaagccccgcctcacttgcagtgtctcttggggacagagcgaccatcagttgccg
agccagcaaatctgtttctgcctctggatacagttatatgcactggtatcaacagaaaccggggcaaccgccaaagttgctcatatacttggc
gtcaaatctcgaatccggtgttcctgcacggttctccggctctggatctggtacagatttcactctcaacatccatcccgtcgaggaggagga
cgctgtaacctactactgtcagcattccagggagcttccttacacatttggcggcggcacaaagctcgagattaaggcggccgcaattgaag
ttatgtatcctcctccttacctagacaatgagaagagcaatggaaccattatccatgtgaaagggaaacacctttgtccaagtcccctatttc
ccggaccttctaagcccttttgggtgctggtggtggttgggggagtcctggcttgctatagcttgctagtaacagtggcctttattattttct
gggtgaggagtaagaggagcaggctcctgcacagtgactacatgaacatgactccccgccgccctgggcccacccgcaagcattaccagccct
atgccccaccacgcgacttcgcagcctatcgctccgctagcggcggaggtggctctggcggtggcggatcagttgttaaacggggcagaa
agaaactcctgtatatattcaaacaaccatttatgagaccagtacaaactactcaagaggaagatggctgtagctgccgatttccagaagaag
aagaaggaggatgtgaactgggcggcggcggcagcggaggcggtggcagcggcggcggcggctctagagtgaagttcagcaggag
cgcagacgcccccgcgtaccagcagggccagaaccagctctataacgagctcaatctaggacgaagagaggagtacgatgttttggaca
agagacgtggccgggaccctgagatggggggaaagccgagaaggaagaaccctcaggaaggcctgtacaatgaactgcagaaagata
agatggcggaggcctacagtgagattgggatgaaaggcgagcgccggaggggcaaggggcacgatggcctttaccagggtctcagtac
agccaccaaggacacctacgacgcccttcacatgcaggccctgccccctcgcactagttaa.

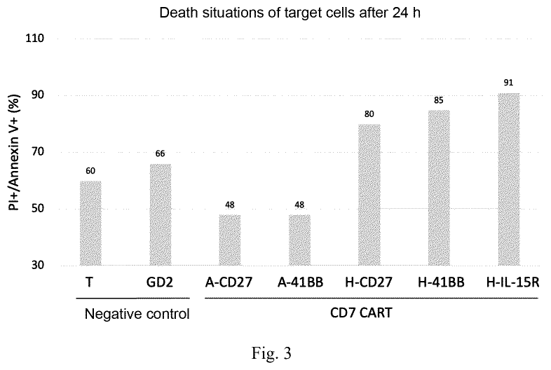

After been killed for 24 h, target cells with green fluorescence were analyzed through flow cytometry and the apoptosis situation of the target cells was quantified through PI/AnnexinV staining. The killing effects of different versions of CD7 CAR-T on Molt-3 were observed as shown in FIG. 3. Compared with two negative control groups, two groups of CD7 CAR-T, A-CD27 and A-41BB, from scFvs in series A have no killing effect on tumor, while the three types of CD7 CAR-T with scFvs in series H in the present application have good killing effects. Therefore, the following clinical trials were continued, and the experiments were independently repeated more than three times. This indicates that not all CD7 scFvs have killing effects.

### Example 6 Clinical Application of CD7 CAR-T Cell Therapy

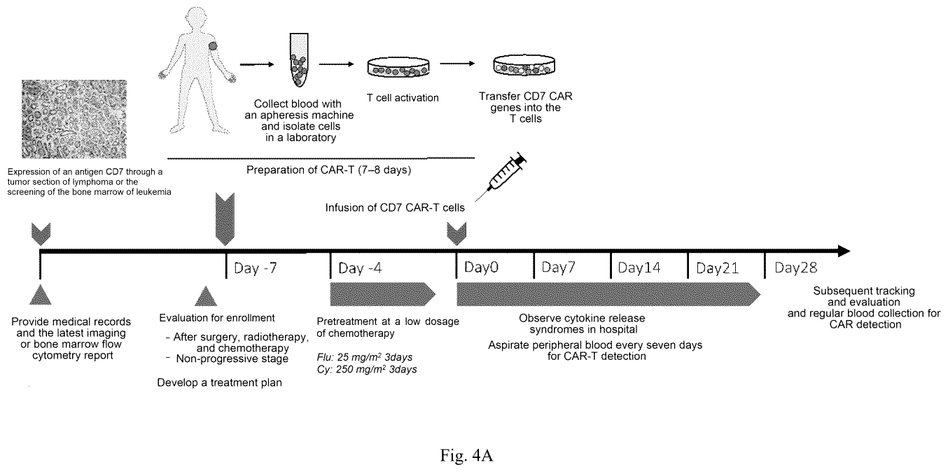

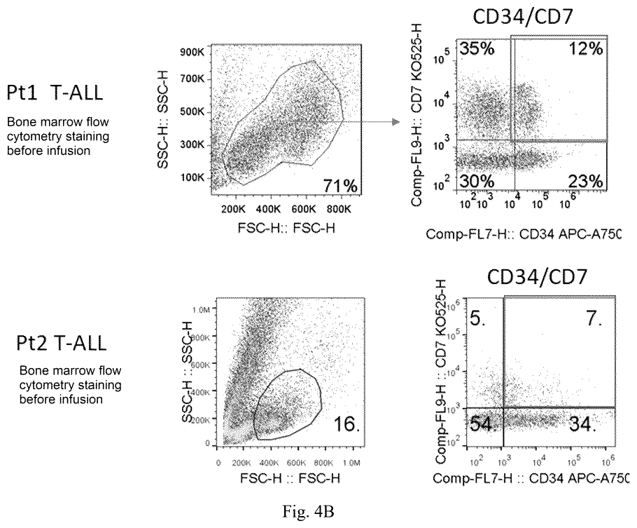

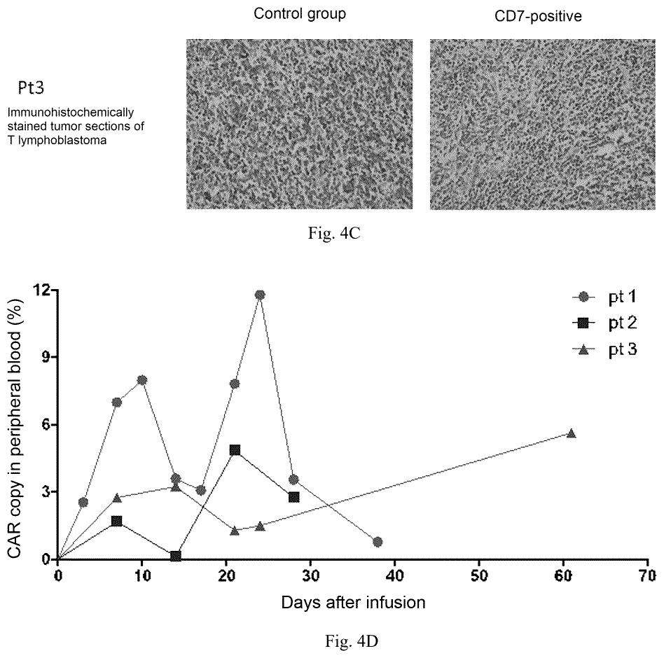

In this example, three types of CD7 CAR-T designed and prepared in the present application were applied to clinical treatment, which includes the steps below.
(1) Two patients with T-ALL and one with T lymphoblastoma were enrolled. The overall treatment process is shown in FIG. 4A and the situations of the patients are summarized in Table 1.
(2) Bone marrow was collected from T-ALL patients prior to enrollment to a laboratory to confirm that the tumors express CD7 (FIG. 4B), and the white slice of the T lymphoblastoma was immunohistochemically stained to confirm the positive expression of CD7 (FIG. 4C).
(3) The white blood cell concentrate was collected from each patient. Peripheral mononuclear lymphocytes in the white blood cell concentrate were separated through density gradient centrifugation with Ficoll, T cells were screened out by CD3 magnetic beads and activated by an anti-CD28 antibody. 2×10⁶ CAR-T cells were prepared per kilogram of the body weight.
(4) Before infused with CAR-T cells, the patients were pretreated with a small dosage of chemotherapy. The pretreatment regimen was cyclophosphamide (250 mg/m²) for three days and fludarabine (25 mg/m²) for three days. CAR-T infusion was conducted 24 h after the pretreatment, which were completed within three days.
(5) H-41BB CAR-T cells were infused through intravenous injection at dosages recorded in Table 1.
(6) After infusion, a clinician monitored the patients and evaluated toxicity. The clinical adverse reactions and cytokine release syndromes (CRSs) of the three patients after infusion are summarized in Table 1, where the three patients are all graded CRS I.
(7) After infusion, a small amount of peripheral blood was periodically aspirated from each patient, peripheral mononuclear lymphocytes were separated, and then cell chromosome DNA (gDNA) was extracted. The CAR copy number in the peripheral blood was quantified through qPCR with specific primers. The variation curves of CAR copy numbers in the three patients are shown in FIG. 4D.
(8) Bone marrow aspirates were extracted by the hospital from two patients with T-ALL before and after CD7 CAR-T infusion, and tumor cells in the bone marrow aspirates were detected. The bone marrow of both two patients was completely remitted after infusion. The details are recorded in Table 1.
(9) The residue, focus, and symptoms of the patient with T lymphoblastoma were evaluated by the hospital before and after CD7 CAR-T infusion. After the CD7 CAR-T infusion, the residue of the cerebrospinal fluid of the patient became negative, and the focus of the brain metastasis had no change, but the symptoms caused by the tumor were improved. The details are recorded in Table 1.

TABLE 1

| | | | Infusion | | | | | |
|---|---|---|---|---|---|---|---|---|
| | | | Dosage | | | | | |
| | | | (Effective | | | | | |
| | | | CAR-T | | | | | |
| | Gender/ | | Number × | Adverse | CRS | Before | After | Clinical |
| No. | Age | Disease | 10\hat{ }6/kg) | Reaction | Grade | Infusion | Infusion | Evaluation |
| pt1 | Male/ | T-ALL | 1.02 | Skin | I | 9.59% | The residue | CR |
| | 11 | | | pruritus, | | residue in | in the bone | |
| | | | | rash, | | the bone | marrow | |
| | | | | and | | marrow | became | |
| | | | | fever | | | negative on | |
| | | | | | | | Day 17 | |
| | | | | | | | (D 17) and | |
| | | | | | | | was | |
| | | | | | | | continuously | |
| | | | | | | | negative | |
| | | | | | | | to D 38 | |
| pt2 | Male/ | T-ALL | 0.74 | Skin | I | 39% | The residue | CR |
| | 59 | | | pruritus | | residue in | in the bone | |
| | | | | and | | the bone | marrow | |
| | | | | fever | | marrow | became | |
| | | | | | | | negative on | |
| | | | | | | | D 31 | |
| pt3 | Male/ | Brain | 2.31 | Skin | I | 896 | The residue | PR |
| | 26 | metastasis of T | | pruritus | | CD7+/CD3 | of the | |
| | | lymphoblastoma | | and | | 4+ cells in | cerebrospinal | |
| | | | | rash | | the | fluid was | |
| | | | | | | cerebrospinal | negative on | |
| | | | | | | fluid | D 31 | |
| | | | | | | Invasion to | The left eye | |
| | | | | | | optic nerves | had no | |
| | | | | | | of the right | change | |
| | | | | | | eye through | through | |
| | | | | | | MRI | MRI | |
| | | | | | | Blurred | The vision | |
| | | | | | | vision of | of the right | |
| | | | | | | the right | eye was | |
| | | | | | | eye | improved | |
| | | | | | | Headache | The | |
| | | | | | | and severe | headache | |
| | | | | | | periocular | and | |
| | | | | | | pain | periocular | |
| | | | | | | | pain were | |
| | | | | | | | relieved | |

Note:
CR, complete remission; PR, partial remission.

In conclusion, the tumor surface antigen CD7 on the tumor cells targeted by the chimeric antigen receptor of the present application is not prone to mutation and has an improved effect as compared with other chimeric antigen receptors and other tumor antigens in targeting T cell cancer. The target-specific CAR is expressed at a high level so that the immune effect and the therapeutic effect of CAR-T cells are enhanced.

The applicant has stated that although the detailed method of the present application is described through the examples described above, the present application is not limited to the detailed method described above, which means that implementation of the present application does not necessarily depend on the detailed method described above. It should be apparent to those skilled in the art that any improvements made to the present application, equivalent replacements of raw materials of the product of the present application, additions of adjuvant ingredients, selections of specific manners, etc., all fall within the protection scope and the disclosure scope of the present application.

1. A CD7-based humanized chimeric antigen receptor, comprising an antigen binding domain, a transmembrane domain, a costimulatory signaling region, and a CD3ζ signaling domain, which are connected in tandem; wherein the antigen binding domain binds to a tumor surface antigen, and the tumor surface antigen is CD7.

2. The CD7-based humanized chimeric antigen receptor of claim 1, wherein the antigen binding domain comprises a humanized CD7 single-chain antibody; preferably, a nucleotide sequence of the humanized CD7 single-chain antibody comprises a sequence having more than 80% homology to a sequence shown in SEQ ID NO. 1; preferably, an amino acid sequence of the humanized CD7 single-chain antibody comprises a sequence having more than 80% homology to a sequence shown in SEQ ID NO. 2; preferably, a nucleotide sequence of the humanized chimeric antigen receptor comprises a sequence having more than 80% homology to a sequence shown in SEQ ID NO. 3, SEQ ID NO. 4, or SEQ ID NO. 5; and preferably, an amino acid sequence of the humanized chimeric antigen receptor comprises a sequence having more than 80% homology to a sequence shown in SEQ ID NO. 6, SEQ ID NO. 7, or SEQ ID NO. 8.

3. The CD7-based humanized chimeric antigen receptor of claim 1, wherein the transmembrane domain is a CD28 transmembrane domain and/or a CD8α transmembrane domain; preferably, the costimulatory signaling region is a combination of a CD28 signaling domain with a 4-1BB, CD27, or IL-15R signaling domain.

4. The CD7-based humanized chimeric antigen receptor of claim 1, wherein the humanized chimeric antigen receptor further comprises a self-destructing domain; preferably, the self-destructing domain comprises a caspase 9 domain; and preferably, the self-destructing domain is connected in tandem with the CD3ζ signaling domain through a 2A sequence.

5. The CD7-based humanized chimeric antigen receptor of claim 1, wherein the humanized chimeric antigen receptor comprises a signal peptide, an antigen binding domain, a transmembrane domain, a costimulatory signaling region, a CD3ζ signaling domain, a 2A sequence, and a self-destructing domain, which are connected in tandem; preferably, the humanized chimeric antigen receptor comprises a Secretory signal peptide, a humanized CD7 single-chain antibody, a CD8α transmembrane domain and/or a CD28 transmembrane domain, a combination of a CD28 signaling domain and a CD27, 4-1BB, or IL-15R signaling domain, a CD3ζ signaling domain, the 2A sequence, and a caspase 9 domain, which are connected in tandem.

6. The CD7-based humanized chimeric antigen receptor of claim 1, wherein the humanized chimeric antigen receptor is Secretory signal peptide-humanized CD7 single-chain antibody-CD28-4-1BB-CD3ζ; preferably, the Secretory signal peptide-humanized CD7 single-chain antibody-CD28-4-1BB-CD3ζ has an ORF nucleic acid sequence as shown in SEQ ID NO. 3; preferably, the humanized chimeric antigen receptor is Secretory signal peptide-humanized CD7 single-chain antibody-CD28-CD27-CD3ζ-2A-iCasp9; preferably, the Secretory signal peptide-humanized CD7 single-chain antibody-CD28-CD27-CD3ζ-2A-iCasp9 has an ORF nucleic acid sequence as shown in SEQ ID NO.4; preferably, the chimeric antigen receptor is Secretory signal peptide-humanized CD7 single-chain antibody-CD28-IL-1 5R-CD3ζ; and preferably, the Secretory signal peptide-humanized CD7 single-chain antibody-CD28-IL-15R-CD3ζ has an ORF nucleic acid sequence as shown in SEQ ID NO. 5.

7. The CD7-based humanized chimeric antigen receptor of claim 1, wherein a method for preparing the humanized chimeric antigen receptor comprises: transducing a nucleic acid sequence encoding the humanized chimeric antigen receptor into a T cell for expression; preferably, the nucleic acid sequence is transduced into the T cell through any one or a combination of at least two of a viral vector, an eukaryotic expression plasmid, or an mRNA sequence; preferably, the nucleic acid sequence is transduced into the T cell through the viral vector; preferably, the viral vector is any one or a combination of at least two of a lentiviral vector or a retroviral vector, preferably a lentiviral vector.

8. A recombinant lentivirus, comprising a vector expressing the CD7-based humanized chimeric antigen receptor of claim 1.

9. A composition, comprising the CD7-based humanized chimeric antigen receptor of claim 1.

10. Use of the chimeric antigen receptor of claim 1, in the preparation of a chimeric antigen receptor T cell or a medicament for treating a tumor; preferably, the tumor is a blood-related tumor disease; and preferably, the blood-related tumor disease is T-cell-associated leukemia or lymphoma.
